(19) United States
(12) Patent Application Publication
(10) Pub. No.: US 2006/0052630 A1
(43) Pub. Date: Mar. 9, 2006

(54) Process for the producing alkylarylsulfonates by using modified, dimerized olefins

(75) Inventors: Thomas Narbeshuber, Mannheim (DE); Ulrich Steinbrenner, Neustadt (DE); Dag Wiebelhaus, Neustadt (DE); Nils Bottke, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 10/538,473

(22) PCT Filed: Dec. 22, 2003
(86) PCT No.: PCT/EP03/14712

§ 371 Date: Jun. 7, 2005

(30) Foreign Application Priority Data
Dec. 23, 2002 (DE) 102 61 481.4

(51) Int. Cl. C07C 303/06

(52) U.S. Cl. 562/81

(57) ABSTRACT

The invention relates to processes for the preparation of alkylarylsulfonates by
a) reaction of a C₄-olefin mixture over a metathesis catalyst to prepare an olefin mixture comprising 2-pentene and/or 3-hexene and optional removal of 2-pentene and/or 3-hexene,
b) dimerization of the 2-pentene and/or 3-hexene obtained in stage a) in the presence of a dimerization catalyst to give a mixture comprising C₁₀₋₁₂-olefins and optional removal of the C₁₀₋₁₂-olefins,
c) reaction of the C₁₀₋₁₂-olefin mixtures obtained in stage b) with an aromatic hydrocarbon in the presence of an alkylation catalyst to form alkyl aromatic compounds, where, prior to the reaction, additional linear olefins may be added, d) sulfonation of the alkyl aromatic compounds obtained in stage c) and neutralization to give alkylarylsulfonates, where, prior to the sulfonation, linear alkylbenzenes may additionally be added, e) optional mixing of the alkylarylsulfonates obtained in stage d) with linear alkylarylsulfonates.

The present invention relates to processes for the preparation of alkylarylsulfonates, to alkylarylsulfonates obtainable by the process, and to alkylaryls obtainable in the process as intermediates, to the use of the alkylarylsulfonates as surfactants, preferably in detergents and cleaners, and to detergents and cleaners comprising them.

Alkylbenzenesulfonates (ABS) have been used for a long time as surfactants and detergents and cleaners. Following the use initially of such surfactants based on tetrapropylene, which, however, had poor biodegradability, predominantly linear alkylbenzenesulfonates (LAS) have been prepared and used in the following period. However, linear alkylbenzenesulfonates do not have properties profiles which are adequate in all fields of use.

Thus, for example, it would be advantageous to improve their low-temperature washing properties or their properties in hard water. Likewise desirable is the ready ability to be formulated, which arises from the viscosity of the sulfonates and their solubility. These improved properties are achieved by slightly branched compounds or mixtures of slightly branched compounds with linear compounds, although the correct degree of branching and/or the correct degree of mixing must be achieved. Excessive branching impairs the biodegradability of the products. Products which are too linear adversely affect the viscosity and the solubility of the sulfonates.

Moreover, the proportion of terminal phenylalkanes (2-phenylalkanes and 3-phenylalkanes) relative to internal phenylalkanes (4-, 5-, 6- etc. phenylalkanes) plays a role for the product properties. A 2-phenyl content of about 30% and a 2- and 3-phenyl content of about 50% can be advantageous with regard to product quality (solubility, viscosity, washing properties).

Surfactants with excessively high 2- and 3-phenyl contents can have the important disadvantage that the processability of the products suffers as a result of a large increase in the viscosity of the sulfonates.

Moreover, this may give rise to nonoptimal solubility behavior. Thus, for example, the Krafft point of a solution of LAS with very high or very low 2- and 3-phenyl contents is around up to 10-20° C. higher than for the optimum choice of the 2- and 3-phenyl content.

The process according to the invention offers the important advantage that by combining metathesis and dimerization, a unique olefin mixture is obtained which, following alkylation of an aromatic, sulfonation and neutralization, produces a surfactant which is characterized by its combination of excellent application properties (solubility, viscosity, stability toward water hardness, washing properties, biodegradability). With regard to the biodegradability of alkylarylsulfonates, compounds which are adsorbed to sewage sludge to a lesser extent than conventional LAS are particularly advantageous.

For this reason, alkylbenzenesulfonates branched to a certain degree have been developed.

WO 99/05241 relates to cleaners which comprise branched alkylarylsulfonates as surfactants. The alkylarylsulfonates are obtained by dimerization of olefins to give vinylidene olefins and subsequent alkylation of benzene over a shape-selective catalyst such as MOR or BEA. This is followed by a sulfonation.

WO 02/44114 relates to a process for the preparation of alkylarylsulfonates in which singly branched C₁₀₋₁₄-olefins obtainable by various processes are reacted with an aromatic hydrocarbon in the presence of a zeolite of the faujasite type as alkylation catalyst. The C₁₀₋₁₄-olefins can be prepared, for example, by metathesis of a C₄-olefin mixture, followed by a dimerization of the resulting 2-pentene and/or 3-hexene over a dimerization catalyst. Alternative processes are extraction, Fischer-Tropsch synthesis, dimerization or isomerization of olefins.

WO 02/14266 relates to a process for the preparation of alkylarylsulfonates in which firstly a metathesis of a C₄-olefin mixture to prepare 2-pentene and/or 3-hexene is carried out, and the products are subjected to a dimerization. An alkylation is then carried out in the presence of an alkylation catalyst, followed by a sulfonation and neutralization.

The olefins used hitherto for the alkylation sometimes have too high or too low a degree of branching, or produce a non-optimum ratio of terminal to internal phenylalkanes. Secondly, they are prepared from expensive starting materials, such as, for example, propene or alpha-olefins, and in some cases the proportion of the olefin fractions of interest for the surfactant preparation is only about 20%. This leads to costly work-up steps. The last-mentioned processes do not lead in all cases to products which exhibit a desired spectrum of properties.

It is an object of the present invention to provide a process for the preparation of alkylarylsulfonates which are at least partially branched and thus have advantageous properties for use in detergents and cleaners compared with the known compounds. In particular, they should have a suitable profile of properties of biodegradability, insensitivity toward water hardness, solubility and viscosity during preparation and during use. In addition, the alkylarylsulfonates should be preparable in a cost-effective manner.

We have found that this object is achieved according to the invention by a process for the preparation of alkylarylsulfonates by
a) reaction of a C₄-olefin mixture over a metathesis catalyst to prepare an olefin mixture comprising 2-pentene and/or 3-hexene, and optional removal of 2-pentene and/or 3-hexene,
b) dimerization of the 2-pentene and/or 3-hexene obtained in stage a) in the presence of a dimerization catalyst to give a mixture comprising C₁₀₋₁₂-olefins, removal of the C₁₀₋₁₂-olefins and removal of 5 to 30% by weight, based on the C₁₀₋₁₂-olefins removed, of low-boiling constituents of the C₁₀₋₁₂-olefins,
c) reaction of the C₁₀₋₁₂-olefin mixtures obtained in stage b) with an aromatic hydrocarbon in the presence of an alkylation catalyst to form alkyl aromatic compounds, where, prior to the reaction, 0 to 60% by weight, preferably 0 to 40% by weight, based on the C₁₀₋₁₂-olefin mixtures obtained in stage b), of linear olefins may additionally be added,
d) sulfonation of the alkyl aromatic compounds obtained in stage c) and neutralization to give alkylarylsulfonates, where, prior to the sulfonation, 0 to 60% by weight, preferably 0 to 50% by weight, based on the alkyl aromatic compounds obtained in stage c), of linear alkylbenzenes may additionally be added, if no admixing has taken place in stage c),
e) optional mixing of the alkylarylsulfonates obtained in stage d) with 0 to 60% by weight, preferably 0 to 30% by weight, based on the alkylarylsulfonates obtained in stage d), of linear alkylarylsulfonates, if no admixing has taken place in stages c) and d),
   and also by a process for the preparation of alkylarylsulfonates by
a) reaction of a C₄-olefin mixture over a metathesis catalyst to prepare an olefin mixture comprising 2-pentene and/or 3-hexene and optional removal of 2-pentene and/or 3-hexene,
b) dimerization of the 2-pentene and/or 3-hexene obtained in stage a) in the presence of a dimerization catalyst to give a mixture comprising C₁₀₋₁₂-olefins and optional removal of the C₁₀₋₁₂-olefins,
c) reaction of the C₁₀₋₁₂-olefin mixtures obtained in stage b) with an aromatic hydrocarbon in the presence of an alkylation catalyst to form alkyl aromatic compounds, where, prior to the reaction, additional linear olefins may be added,
d) sulfonation of the alkyl aromatic compounds obtained in stage c) and neutralization to give alkylarylsulfonates, where, prior to the sulfonation, linear alkylbenzenes may additionally be added,
e) optional mixing of the alkylarylsulfonates obtained in stage d) with linear alkylarylsulfonates,
   where, in at least one of stages c), d) and e), 5 to 60% by weight, in each case based on the mixtures obtained in the previous stage, of the linear compounds are added and the sum of the additions is not more than 80% by weight, preferably not more than 60%, particularly preferably not more than 50% by weight.

The combination of a metathesis of C₄-olefins with a subsequent dimerization and alkylation of aromatic hydrocarbons permits, under said conditions, the use of cost-effective starting materials and of preparation processes which makes the desired products accessible in high yields.

It has been found according to the invention that the metathesis of C₄-olefins gives products which can be dimerized to slightly branched C₁₀₋₁₂-olefin mixtures. By adjusting the desired degree of branching, for example by selective dimerization or removal of a low-boiling fraction and/or addition of linear olefins, these mixtures can be used advantageously in the alkylation of aromatic hydrocarbons, giving products which, following sulfonation and neutralization, produce surfactants which have excellent properties, in particular with regard to sensitivity towards hardness-forming ions, solubility of the sulfonates, viscosity of the sulfonates and their washing properties. Moreover, the present process is extremely cost-effective since the product streams can be arranged so flexibly that no by-products are produced. Starting from a C₄ stream, the metathesis according to the invention produces linear, internal olefins which are then converted into branched olefins via the dimerization step.

Stage a) of the process according to the invention is the reaction of a C₄-olefin mixture over a metathesis catalyst to prepare an olefin mixture comprising 2-pentene and/or 3-hexene, and optional removal of 2-pentene and/or 3-hexene. The metathesis can be carried out, for example, as described in WO 00/39058 or DE-A-100 13 253.

The olefin metathesis (disproportionation) describes, in its simplest form, the reversible, metal-catalyzed transalkylidenation of olefins as a result of breakage or new formation of C═C double bonds in accordance with the following equation: 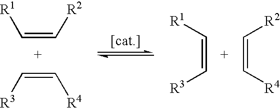
In the special case of the metathesis of acyclic olefins, a distinction is made between self-metathesis, in which an olefin converts to a mixture of two olefins of different molar mass (for example: propene→ethene+2-butene), and cross- or co-metathesis, which describes a reaction of two different olefins (propene+1-butene→ethene+2-pentene). If one of the reactants is ethene, then ethenolysis is generally the term used.

Suitable metathesis catalysts are in principle homogeneous and heterogeneous transition metal compounds, in particular those of subgroup VI to VIII of the Periodic Table of the Elements, and also homogeneous and heterogeneous catalyst systems in which these compounds are present.

Various metathesis processes which start from C₄ streams can be used according to the invention.

DE-A-199 32 060 relates to a process for the preparation of C₅-/C₆-olefins by reaction of a starting stream which comprises 1-butene, 2-butene and isobutene, to give a mixture of C₂₋₆-olefins. In the process, propene in particular is obtained from butenes. Additionally, hexene and methylpentene are discharged as products. In the metathesis, no ethene is added. Optionally, ethene formed in the metathesis is recycled to the reactor.

A preferred process for the preparation of optionally propene and hexene from a raffinate II starting stream comprising olefinic C₄ hydrocarbons comprises
a) in the presence of a metathesis catalyst, which comprises at least one compound of a metal of subgroup VIb, VIIb or VIII of the Periodic Table of the Elements, carrying out a metathesis reaction, in the course of which butenes present in the starting stream are reacted with ethene to give a mixture comprising ethene, propene, butenes, 2-pentene, 3-hexene and butanes, where, based on the butenes, up to 0.6 mol equivalents of ethene may be used,
b) separating the resulting exit stream initially by distillation into optionally a low-boiling fraction A comprising C₂-C₃-olefins, and also into a high-boiling fraction comprising C₄-C₆-olefins and butanes,
c) then separating the low-boiling fraction A optionally obtained from b) by distillation into an ethene-containing fraction and a propene-containing fraction, where the ethene-containing fraction is recycled to process step a) and the propene-containing fraction is discharged as product,
d) then separating the high-boiling fraction obtained from b) by distillation into a low-boiling fraction B comprising butenes and butanes, a medium-boiling fraction C comprising 2-pentene and into a high-boiling fraction D comprising 3-hexene,
e) where the fractions B and optionally C are completely or partially recycled to process step a), and fraction D and optionally C are discharged as product.

An alternative preferred process for the preparation of C₆-alkenes from a hydrocarbon stream comprising C₄-alkenes (starting stream C₄⁼) comprises
a) in a step a), bringing the stream C₄⁼ into contact with a metathesis catalyst which comprises at least one compound of a metal of subgroup VIIb, VIIb or VIII of the Periodic Table of the Elements, where at least part of the C₄-alkenes is reacted to C₂-C₆-alkenes, and the material stream comprising the C₂-C₆-alkenes formed in the process (stream C₂₋₆⁼) is separated off from the metathesis catalyst,
b) in a step b), removing ethylene by distillation from the stream C₂₋₆⁼ and thus preparing a material stream comprising C₃- to C₆-alkenes (stream C₃₋₆⁼) and preparing a material stream consisting essentially of ethylene (stream C₂⁼),
c) in a step c), separating the stream C₃₋₆⁼ by distillation into a material stream consisting essentially of propylene (stream C₃⁼), a material stream consisting essentially of C₆-alkenes (stream C₆⁼) and one or more material streams, chosen from the following group: a material stream consisting essentially of C₄-alkenes (stream C₄⁼), a material stream consisting essentially of C₅-alkenes (stream C₅⁼) and a material stream consisting essentially of C₄- and C₅-alkenes (stream C₄₋₅⁼),
d) in a step d), using one or more material streams or parts thereof, chosen from the group stream C₄⁼, stream C₅⁼ and stream C₄₋₅⁼, completely or partially for the preparation of starting stream C₄⁼ (recycle stream), and optionally discharging the stream(s), or the part(s) thereof, which are not recycle stream.

The starting stream C₄⁼ is subjected here to a metathesis reaction in accordance with a process as described in EP-A 1069101.

The metathesis reaction according to step a) is carried out here preferably in the presence of heterogeneous metathesis catalysts which are not or only slightly isomerization-active and which are chosen from the class of transition metal compounds of metals of group VIIb, VIIb or VIII of the Periodic Table of the Elements applied to inorganic supports.

As metathesis catalyst, preference is given to using rhenium oxide on a support, preferably on γ-aluminum oxide or on Al₂O₃/B₂O₃/SiO₂ mixed supports.

In particular, the catalyst used is Re₂O₇/γ-Al₂O₃ with a rhenium oxide content of from 1 to 20% by weight, preferably 3 to 15% by weight, particularly preferably 6 to 12% by weight.

The metathesis is carried out in the liquid procedure preferably at a temperature of from 0 to 150° C., particularly preferably 20 to 80° C., and a pressure of from 2 to 200 bar, particularly preferably 5 to 30 bar.

If the metathesis is carried out in the gas phase, the temperature is preferably 20 to 300° C., particularly preferably 50 to 200° C. The pressure in this case is preferably 1 to 20 bar, particularly preferably 1 to 5 bar. Detailed information regarding the metathesis reaction is given again in EP-A 1069101.

The subsequent work-up of the stream C₂₋₆⁼ formed in the metathesis takes place in steps b) and c) described at the outset.

Preferably, in step c), the procedure is in accordance with the 3 following alternative processes:

Variant 1 is carried out in the form of 2 partial steps c1) and c2) usually in two separate columns by
c1) in step c1), separating the stream C₃₋₆⁼ by distillation into a material stream consisting essentially of propylene (stream C₃⁼), usually as head take-off, and a material stream consisting essentially of C₄-alkenes, C₅- and C₆-alkenes (stream C₄₋₆⁼), usually as bottom take-off, and
c2) in step c2), separating the stream C₄₋₆⁼ by distillation into a material stream consisting essentially of butenes (stream C₄⁼), usually as head take-off, and a material stream consisting essentially of C₄- and C₅-alkenes (stream C₄₋₅⁼), usually as head take-off, and a stream C₆⁼ [variant CS]
   Variant 2 is carried out in the form of 2 partial steps c3) and c4) usually in two separate columns by
c3) in step c3), separating the stream C₃₋₆⁼ by distillation into a material stream consisting essentially of C₆-alkenes (stream C₆⁼), usually as bottom take-off, and a material stream consisting essentially of propene, butenes and C₅-alkenes (stream C₄₋₅⁼), usually as head take-off, and
c4) in step c4), separating the stream C₄₋₅⁼ by distillation into a material stream consisting essentially of propene (stream C₃⁼), usually as head take-off, a material stream consisting essentially of C₄-alkenes (stream C₄⁼), usually as side take-off, and a material stream consisting essentially of butenes and C₅-alkenes (stream C₄₋₅⁼), usually as bottom take-off. [Variant DS]
   Variant 3 is carried out in the form of 3 partial steps c5) to c7) usually in three separate columns by
c5) in step c5), separating the stream C₃₋₆⁼ by distillation into a material stream consisting essentially of propene and C₄-alkenes (stream C₃₋₄⁼), usually as head take-off, and a material stream consisting essentially of C₅- and C₆-alkenes (stream C₅₋₆⁼), usually as bottom take-off, and
c6) in step c6), separating the stream C₃₋₄⁼ by distillation into a stream C₃⁼, usually as head take-off, and a stream C₄⁼, usually as bottom take-off.
c7) in step c7), separating the stream C₅₋₆⁼ by distillation into a stream C₅⁼, usually as head take-off, and a stream C₆⁼, usually as bottom take-off. [Variant F]

The separation efficiency of the columns is generally adjusted such that propene and ethylene are obtained with a purity of more than 99% by weight.

The streams C₄⁼, C₅⁼ and C₄₋₅⁼ formed in the individual variants of step c) are used, in part or in their entirety, as recycle stream according to step d), as already described above, for the preparation of the starting stream C₄⁼.

The proportion of material streams C₄⁼, C₅⁼ and C₄₋₅⁼ which is used as recycle stream is usually 10 to 70%, based on the sum of recycle stream and discharged fraction of the material streams C₄⁼, C₅⁼ and C₄₋₅⁼. If, in material stream C₄⁼ or C₄⁼, C₄-alkanes are also present as well as 1- and 2-butene and also isobutene, at least some of the C₄-alkanes must be removed in order to avoid a concentration of the C₄-alkanes, or it is possible to use only some of these streams as recycle stream.

The individual streams and fractions can comprise said compounds or consist of them. In the event that they consist of the streams or compounds, the presence of relatively small amounts of other hydrocarbons is not ruled out.

In this process, in a single-stage reaction procedure in a metathesis reaction, a fraction consisting of C₄-olefins, preferably n-butenes, and butanes is reacted optionally with variable amounts of ethene over a homogeneous or preferably heterogeneous metathesis catalyst to give a product mixture of (inert) butanes, unreacted 1-butene, 2-butene and also the metathesis products ethene, propene, 2-pentene and 3-hexene. The desired products 2-pentene and/or 3-hexene are discharged, and the products which remain and unreacted compounds are completely or partially recycled to the metathesis. Preferably, they are recycled as completely as possible, with only small amounts being discharged in order to avoid an accumulation. Ideally, no accumulation results and all of the compounds apart from 3-hexene are recycled to the metathesis.

According to the invention, based on the butenes in the C₄ feed stream, up to 0.6, preferably up to 0.5, mol equivalents of ethene are used. Thus, compared with the prior art, only small amounts of ethene are used.

In addition, according to the invention the maximum possible amounts of C₄ products and optionally C₅ products present in the reactor discharge are recycled. This relates in particular to the recycling of unreacted 1-butene and 2-butene and also to any 2-pentene formed.

If small amounts of isobutene are still present in the C₄ feed stream, small amounts of branched hydrocarbons may also be formed.

The amount of branched C₅- and C₆-hydrocarbons possibly additionally formed in the metathesis discharge is dependent on the isobutene content in the C₄ feed and is preferably kept as low as possible (<3%).

In order to illustrate the process according to the invention in several variations in more detail, the reaction which takes place in the metathesis reactor is divided into three important individual reactions:
1. Cross-Methathesis of 1-Butene with 2-Butene 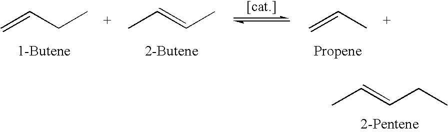
2. Self-Metathesis of 1-Butene 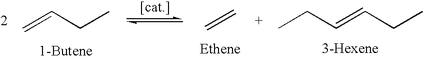
3. Optional Ethenolysis of 2-Butene 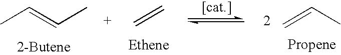
Depending on the respective requirement for the target products propene and 3-hexene (the term 3-hexene includes inter alia any isomers formed) or 2-pentene, the external mass balance of the process can be influenced in a targeted manner through the variable use of ethene and by shifting the equilibrium by recycling certain partial streams. Thus, for example, the 3-hexene yield can be increased by suppressing the cross-metathesis of 1-butene with 2-butene by recycling 2-pentene to the metathesis step, meaning that here no or the smallest possible amount of 1-butene is consumed. During the self-metathesis of 1-butene to 3-hexene which then preferably takes place, ethene is additionally formed which reacts in a subsequent reaction with 2-butene to give the product-of-value propene.

Olefin mixtures which comprise 1-butene and 2-butene and optionally isobutene are obtained inter alia in diverse cracking processes, such as steam cracking or FCC cracking, as C₄ fraction. Alternatively, it is possible to use butene mixtures as are produced in the dehydrogenation of butanes or by dimerization of ethene. Butanes present in the C₄ fraction have inert behavior. Dienes, alkynes or enynes are removed prior to the metathesis step according to the invention using customary methods such as extraction or selective hydrogenation.

The butene content of the C₄ fraction used in the process is 1 to 100% by weight, preferably 60 to 90% by weight. The butene content refers here to 1-butene, 2-butene and isobutene.

Preference is given to using a C₄ fraction as is produced during steam cracking or FCC cracking or during the dehydrogenation of butane.

Here, the C₄ fraction used is preferably raffinate II, where the C₄ stream is freed from troublesome impurities prior to the metathesis reaction by appropriate treatment over adsorber protection beds, preferably over high-surface-area aluminum oxides or molecular sieves.

In step d), the fractionation into low-boiling fraction B, medium-boiling fraction C and high-boiling fraction D can, for example, be carried out in a dividing-wall column. Here, the low-boiling fraction B is obtained overhead, the medium-boiling fraction C is obtained via a mid-discharge and the high-boiling fraction D is obtained as the bottom product.

The metathesis reaction is here preferably carried out in the presence of heterogeneous metathesis catalysts which are not or only slightly isomerization-active and which are chosen from the class of transition metal compounds of metals of group VIb, VIIb or VIII of the Periodic Table of the Elements applied to inorganic supports.

Preferably, the metathesis catalyst used is rhenium oxide on a support, preferably on γ-aluminum oxide or on Al₂O₃/B₂O₃/SiO₂ mixed supports.

In particular, the catalyst used is Re₂O₇/γ-Al₂O₃ with a rhenium oxide content of from 1 to 20% by weight, preferably 3 to 15% by weight, particularly preferably 6 to 12% by weight.

The metathesis is carried out in the liquid procedure preferably at a temperature of from 0 to 150° C., particularly preferably 20 to 110° C., and a pressure of from 2 to 200 bar, particularly preferably 5 to 40 bar.

If the metathesis is carried out in the gas phase, the temperature is preferably 20 to 300° C., particularly preferably 50 to 200° C. The pressure in this case is preferably 1 to 20 bar, particularly preferably 1 to 5 bar.

The preparation of C₅/C₆-olefins and optionally propene from steam cracker or refinery C₄ streams can include the partial steps (1) to (4):
(1) removal of butadiene and acetylenic compounds by optional extraction of butadiene with a butadiene-selective solvent and subsequently/or selective hydrogenation of butadienes and acetylenic impurities present in the crude C₄ cut in order to obtain a reaction discharge which comprises n-butenes and isobutene and essentially no butadienes and acetylenic compounds,
(2) removal of isobutene by reaction of the reaction discharge obtained in the preceding stage with an alcohol in the presence of an acidic catalyst to give an ether, removal of the ether and of the alcohol, which can take place simultaneously or after the etherification, in order to obtain a reaction discharge which comprises n-butenes and optionally oxygenate impurities, where ether formed can be discharged or back-cleaved to obtain pure isobutene, and the etherification step can be followed by a distillation step to remove isobutene, where optionally also entrained C₃—, i-C₄- and C₅-hydrocarbons can be removed by distillation in the course of working-up the ether, or oligomerization or polymerization of isobutene from the reaction discharge obtained in the preceding stage in the presence of an acidic catalyst whose acid strength is suitable for the selective removal of isobutene as oligo- or polyisobutene in order to obtain a stream which has 0 to 15% of residual isobutene,
(3) removal of the oxygenate impurities from the discharge of the preceding steps over appropriately chosen adsorber materials,
(4) metathesis reaction of the resulting raffinate II stream as described.

Preferably, the partial step of selective hydrogenation of butadiene and acetylenic impurities present in crude C₄ cut is carried out in two stages by bringing the crude C₄ cut in liquid phase into contact with a catalyst which comprises at least one metal, chosen from the group consisting of nickel, palladium and platinum, on a support, preferably palladium on aluminum oxide, at a temperature of from 20 to 200° C., a pressure of from 1 to 50 bar, a liquid hourly space velocity of from 0.5 to 30 m³ of fresh feed per m³ of catalyst per hour and a ratio of recycle to feed stream of from 0 to 30 with a molar ratio of hydrogen to diolefins of from 0.5 to 50 in order to obtain a reaction discharge in which, as well as isobutene, the n-butenes 1-butene and 2-butene are present in a molar ratio of from 2:1 to 1:10, preferably from 2:1 to 1:3, and essentially no diolefins and acetylenic compounds are present. For a maximum hexene discharge, 1-butene is preferably in excess; for a high propene yield, 2-butene is preferably in excess. This means that the overall molar ratio in the first case may be 2:1 to 1:1 and in the second case 1:1 to 1:3.

The partial step of butadiene extraction from crude C₄ cut is preferably carried out with a butadiene-selective solvent chosen from the class of polar-aprotic solvents, such as acetone, furfural, acetonitrile, dimethylacetamide, dimethylformamide and N-methylpyrrolidone, in order to obtain a reaction discharge in which, following subsequent selective hydrogenation/isomerization, the n-butenes 1-butene and 2-butene are present in a molar ratio 2:1 to 1:10, preferably from 2:1 to 1:3.

The partial step of isobutene etherification is preferably carried out in a three-stage reactor cascade with methanol or isobutanol, preferably isobutanol in the presence of an acidic ion exchanger, in which flooded fixed-bed catalysts are passed through from top to bottom, where the reactor inlet temperature is 0 to 60° C., preferably 10 to 50° C., the outlet temperature is 25 to 85° C., preferably 35 to 75° C., the pressure is 2 to 50 bar, preferably 3 to 20 bar and the ratio of isobutanol to isobutene is 0.8 to 2.0, preferably 1.0 to 1.5, and the overall conversion corresponds to the equilibrium conversion.

The partial step of isobutene removal by oligomerization or polymerization of isobutene starting from the reaction discharge obtained by the above-described stages of butadiene extraction and/or selective hydrogenation is preferably carried out in the presence of a catalyst chosen from the class of homogeneous and heterogeneous Broensted or Lewis acids, see DE-A-100 13 253.

Selective Hydrogenation of Crude C₄ Cut

Alkynes, alkynenes and alkadienes are undesired substances in many industrial syntheses due to their tendency toward polymerization or their marked tendency for complexation with transition metals. They sometimes very severely adversely affect the catalysts used in these reactions.

The C₄ stream of a steam cracker comprises a high fraction of polyunsaturated compounds such as 1,3-butadiene, 1-butyne (ethylacetylene) and butenyne (vinylacetylene). Depending on the downstream processing present, the polyunsaturated compounds are either extracted (butadiene extraction) or selectively hydrogenated. In the first-mentioned case, the residual content of polyunsaturated compounds is typically 0.05 to 0.3% by weight, and in the last-mentioned case it is typically 0.1 to 4.0% by weight. Since the residual amounts of polyunsaturated compounds are likewise undesired during the further processing, a further concentration by selective hydrogenation to values of <10 ppm is required. In order to obtain the highest possible product-of-value fraction of butenes, the overhydrogenation to butanes must be kept as low as possible.

Alternative: Extraction of Butadiene from Crude C₄ Cut

The preferred process for butadiene isolation is based on the physical principle of extractive distillation. As a result of the addition of selective organic solvents, the volatility of specific components of a mixture, in this case butadiene, is lowered. These therefore remain with the solvent in the bottom of the distillation column, while the accompanying substances which could not previously be separated off by distillation can be removed overhead. The solvents used for the extractive distillation are mainly acetone, furfural, acetonitrile, dimethylacetamide, dimethylformamide (DMF) and N-methylpyrrolidone (NMP). Extractive distillations are suitable particularly for butadiene-rich C₄ cracker cuts with a relatively high content of alkynes, including methyl-, ethyl- and vinylacetylene, and also methylallene.

The simplified principle of a solvent extraction from crude C₄ cut can be represented as follows: the completely evaporated C₄ cut is introduced into an extraction column at the lower end. The solvent (DMF, NMP) flows from the top in the opposite direction to the gas mixture and on the way down becomes laden with better-soluble butadiene and small amounts of butenes. At the lower end of the extraction column, some of the pure butadiene obtained is introduced in order to expel the butenes as far as possible. The butenes leave the fractionating column at the top. In a further column, referred to as degasser, the butadiene is freed from the solvent by boiling out and then purified by distillation.

Usually, the reaction discharge from a butadiene extractive distillation is fed to the second stage of a selective hydrogenation in order to reduce the butadiene residual content to values of <10 ppm.

The C₄ stream which remains following the removal of butadiene is referred to as the C₄ raffinate or raffinate I and comprises mainly the components isobutene, 1-butene, 2-butenes, and n- and isobutanes.

Removal of Isobutene from Raffinate I

During the further fractionation of the C₄ stream, isobutene is preferably subsequently isolated since it differs from the other C₄ components by virtue of its branching and its higher reactivity. As well as the possibility of a shape-selective molecular sieve separation, with which isobutene can be obtained with a purity of 99%, and n-butenes and butane adsorbed to the molecular sieve pores can be desorbed again by means of a higher-boiling hydrocarbon, this is carried out primarily by distillation using a deisobutenizer, with which isobutene is removed overhead together with 1-butene and isobutene, and 2-butenes and also n-butane including residual amounts of iso- and 1-butene remain in the still, or by extraction by reaction of isobutene with alcohols over acidic ion exchangers. For this, preference is given to using methanol (→MTBE) or isobutanol (IBTBE).

The preparation of MTBE from methanol and isobutene takes place at 30 to 100° C. and slight superatmospheric pressure in the liquid phase over acidic ion exchangers. The procedure is either carried out in two reactors or in a two-stage shaft reactor in order to achieve a virtually complete isobutene conversion (>99%). The pressure-dependent azeotrope formation between methanol and MTBE requires, for the synthesis of pure MTBE, a multistage pressurized distillation or is achieved in accordance with newer technology by methanol adsorption on adsorber resins. All other components of the C₄ fraction remain unchanged. Since small fractions of diolefins and acetylenes can shorten the lifespan of the ion exchanger as a result of polymer formation, preference is given to using bifunctional PD-containing ion exchangers in which only diolefins and acetylenes are hydrogenated in the presence of small amounts of hydrogen. The etherification of the isobutene remains uninfluenced by this.

MTBE is used primarily to increase the octane number of motor gasoline. MTBE and IBTBE can alternatively be back-cleaved over acidic oxides in the gas phase at 150 to 300° C. to obtain pure isobutene.

A further way of removing isobutene from raffinate I consists in the direct synthesis of oligo/polyisobutene. Over acidic homogeneous and heterogeneous catalysts, such as, for example, tungsten trioxide on titanium dioxide, it is possible in this way to obtain, at isobutene conversions up to 95%, a discharge stream which has a residual content of isobutene of at most 5%.

Feed Purification of the Raffinate II Stream Over Adsorber Materials

To improve the service life of the catalysts used for the subsequent metathesis step, as described above, the use of a feed purification (guard bed) is required to remove catalyst poisons, such as, for example, water, oxygenates, sulfur or sulfur compounds or organic halides.

Processes for adsorption and adsorptive purification are described, for example, in W. Kast, Adsorption aus der Gasphase [Adsorption from the Gas Phase], VCH, Weinheim (1988). The use of zeolitic adsorbents is explained in D. W. Breck, Zeolite Molecular Sieves, Wiley, New York (1974).

The removal of acetaldehyde in particular from C₃— to C₁₋₅-hydrocarbons in the liquid phase can take place in accordance with EP-A-0 582 901.

Selective Hydrogenation of Crude C₄ Cut

From the crude C₄ fraction originating from a steam cracker or a refinery is firstly selectively hydrogenated butadiene (1,2- and 1,3-butadiene), and alkynes or alkenynes present in the C₄ cut, in a two-stage process. The C₄ stream originating from the refinery can, according to one embodiment, also be fed directly to the second step of the selective hydrogenation.

The first step of the hydrogenation is preferably carried out over a catalyst which comprises 0.1 to 0.5% by weight of palladium on aluminum oxide as support. The reaction is operated in gas/liquid phase in a fixed bed (trickle procedure) with a liquid cycle. The hydrogenation takes place at a temperature in the range 40 to 80° C. and a pressure from 10 to 30 bar, a molar ratio of hydrogen to butadiene of from 10 to 50 and a liquid hourly space velocity LHSV of up to 15 m³ of fresh feed per m³ of catalyst per hour and a ratio of recycle of feed stream of 5 to 20.

The second step of the hydrogenation is preferably carried out over a catalyst which comprises 0.1 to 0.5% by weight of palladium on aluminum oxide as support. The reaction is operated in gas/liquid phase in a fixed bed (trickle procedure) with a liquid cycle. The hydrogenation takes place at a temperature in the range from 50 to 90° C. and a pressure from 10 to 30 bar, a molar ratio of hydrogen to butadiene of 1.0 to 10 and a liquid hourly space velocity of from 5 to 20 m³ of fresh feed per m³ of catalyst per hour and a ratio of recycle to feed stream of 0 to 15.

The resulting reaction discharge is referred to as raffinate I and, as well as isobutene, has 1-butene and 2-butene in a molar ratio of 2:1 to 1:10, preferably from 2:1 to 1:3.

Alternative: Removal of Butadiene from Crude C₄ Cut Via Extraction

The extraction of butadiene from crude C₄ cut takes place using N-methylpyrrolidone.

The reaction discharge from the extraction is, according to one embodiment of the invention, fed to the second step of the above-described selective hydrogenation in order to removal residual amounts of butadiene, where in this selective hydrogenation step the desired ratio of 1-butene to 2-butene is set.

Removal of Isobutene Via Etherification with Alcohols

In the etherification stage, isobutene is reacted with alcohols, preferably with isobutanol, over an acidic catalyst, preferably over an acidic ion exchanger, to give ethers, preferably isobutyl tert-butyl ether. The reaction takes place, according to one embodiment of the invention, in a three-stage reactor cascade in which flooded fixed-bed catalysts are passed through from top to bottom. In the first reactor, the inlet temperature is 0 to 60° C., preferably 10 to 50° C.; the outlet temperature is between 25 and 85° C., preferably between 35 and 75° C., and the pressure is 2 to 50 bar, preferably 3 to 20 bar. For a ratio of isobutanol to isobutene of from 0.8 to 2.0, preferably 1.0 to 1.5, the conversion is between 70 and 90%.

In the second reactor, the inlet temperature is 0 to 60° C., preferably 10 to 50° C.; the outlet temperature is between 25 and 85, preferably between 35 and 75° C.; and the pressure is 2 to 50 bar, preferably 3 to 20 bar. The overall conversion over the two stages increases to 85 to 99%, preferably 90 to 97%.

In the third and largest reactor, at the same inlet and outlet temperature of 0 to 60° C., preferably 10 to 50° C., the equilibrium conversion is achieved. The etherification and removal of the ether formed is followed by the ether cleavage: the endothermic reaction is carried out over acidic catalysts, preferably over acidic heterogeneous catalysts, for example phosphoric acid on an SiO₂ support, at an inlet temperature of from 150 to 300° C., preferably at 200 to 250° C., and an outlet temperature of from 100 to 250° C., preferably at 130 to 220° C.

In the event of the use of FCC C₄ cut, it must be taken into account that propane is incorporated in amounts around 1% by weight, isobutene is incorporated in amounts around 30 to 40% by weight, and C₅-hydrocarbons in amounts around 3 to 10% by weight, which can adversely affect the subsequent process sequence. Accordingly, within the scope of the work-up of the ether, the possibility of removal of said components by distillation is provided.

The resulting reaction discharge, referred to as raffinate II, has an isobutene residual content of from 0.1 to 3% by weight.

If the amounts of isobutene in the discharge are relatively large, such as, for example, in the case of the use of FCC C₄ fractions or in the case of the removal of isobutene by acid-catalyzed polymerization to give polyisobutene (partial conversion), the raffinate stream which remains can, according to one embodiment of the invention, be worked-up by distillation prior to the further processing.

Purification of the Raffinate H Stream Over Adsorber Materials

The raffinate II stream obtained following the etherification/polymerization (or distillation) is purified over at least one guard bed consisting of high-surface-area aluminum oxides, silica gels, alumosilicates or molecular sieves. The guard bed here serves to dry the C₄ stream and to remove substances which can act as catalyst poison in the subsequent metathesis step. The preferred adsorber materials are Selexsorb CD and CDO and also 3 Å- and NaX molecular sieves (13×). The purification takes place in drying towers at temperatures and pressures which are chosen such that all of the components are in the liquid phase. Optionally, the purification step is used for prewarming the feed for the subsequent metathesis step.

The raffinate II stream which remains is virtually free from water, oxygenates, organic chlorides and sulfur compounds.

If the etherification step is carried out with methanol to prepare MTBE, it may be necessary, due to the formation of dimethyl ether as secondary component, to combine or connect in series two or more purification steps.

The metathesis catalysts are preferably heterogeneous rhenium catalysts known from the literature, such as Re₂O₇ on γ-A₂O₃ or on mixed supports, such as, for example, SiO₂/Al₂O₃, B₂O₃/SiO₂/Al₂O₃ or Fe₂O₃/Al₂O₃ with varying metal content. The rhenium oxide content is, irrespective of the support chosen, between 1 and 20%, preferably between 3 and 10%.

The catalysts are used in freshly calcined state and require no further activation (e.g. by alkylating agents). Deactivated catalyst can be regenerated a number of times by burning off coke residues at temperatures above 400° C. in a stream of air and cooling under an inert gas atmosphere.

A comparison of the heterogeneous catalysts with one another shows that Re₂O₇/Al₂O₃ is active even under very mild reaction conditions (T=20 to 80° C.), while MO₃/SiO₂ (M=Mo, W) only develops activity at temperatures above 100 to 150° C. and consequently C═C double-bond isomerization can arise as secondary reactions.

Also to be mentioned are:
WO₃/SiO₂, prepared from (C₅H₅)W(CO)₃C1 and SiO₂ in J. Mol. Catal. 1995, 95, 75-83;
3-component system consisting of [Mo(NO)₂(OR)₂]ₙ, SnEt₄ and AlCl₃ in J. Mol. Catal. 1991, 64, 171-178 and J. Mol. Catal 1989, 57, 207-220;
nitridomolybdenum(VI) complexes of highly active precatalysts in J. Organomet. Chem. 1982, 229, C₁₉-C₂₃;
heterogeneous SiO₂—supported MoO₃ and WO₃ catalysts in J. Chem. Soc., Faraday Trans./1982, 78, 2583-2592;
supported Mo catalysts in J. Chem. Soc., Faraday Trans./1981, 77, 1763-1777;
active tungsten catalyst precursor in J. Am. Chem. Soc. 1980, 102(21), 6572-6574;
acetonitrile(pentacarbonyl)tungsten in J. Catal. 1975, 38, 482-484;
trichloro(nitrosyl)molybdenum(II) as catalyst precursor in Z. Chem. 1974, 14, 284-285;
W(CO)₅PPH₃/EtAlCl₂ in J. Catal. 1974, 34, 196-202;
WCl₆/n-BuLi in J. Catal 1973, 28, 300-303;
WCl₆/n-BuLi in J. Catal. 1972, 26, 455-458;

FR 2 726 563: O₃ReO[Al(OR)(L)xO]nReO₃ where R═C₁-C₄₀-hydrocarbon, n=1-10, x=0 or 1 and L=solvent,

EP-A-191 0 675, EP-A-129 0 474, BE 899897: catalyst systems of tungsten, 2-substituted phenoxide radicals and 4 other ligands, including a halogen, alkyl and carbene group.

FR 2 499 083: catalyst system of a tungsten, molybdenum or rhenium oxo transition metal complex with a Lewis acid.

U.S. Pat. No. 4,060,468: catalyst system of a tungsten salt, an oxygen-containing aromatic compound, e.g. 2,6-dichlorophenol and if desired molecular oxygen.

BE 776,564: catalyst system of a transition metal salt, an organometallic compound and an amine.

To improve the cycle life of the catalysts used, primarily of the supported catalysts, the use of a feed purification over adsorber beds (guard beds) is recommended. The guard bed serves here to dry the C₄ stream and to remove substances which may act as catalyst poison in the subsequent metathesis step. The preferred adsorber materials are Selexsorb CD and CDO and also 3 Å and NaX molecular sieves (13×). The purification takes place in drying towers at temperatures and pressures which are preferably chosen such that all of the components are present in the liquid phase. Optionally, the purification step is used for prewarming the feed for the subsequent metathesis step. It may be advantageous to combine or connect in series two or more purification steps.

Pressure and temperature in the metathesis step are chosen such that all of the reactants are in the liquid phase (usually T=0 to 150° C., preferably 20 to 80° C.; p=2 to 200 bar). Alternatively, though, it may be advantageous, particularly in the case of feed streams with a relatively high isobutene content, to carry out the reaction in the gas phase and/or to use a catalyst which has a lower acidity.

As a rule, the reaction is complete after 1 s to 1 h, preferably after 30 s to 30 min. It can be carried out continuously or batchwise in reactors, such as pressurized gas vessels, flow tubes or reactive distillation devices, preference being given to flow tubes.

Stage b)

In stage b) the 2-pentene and/or 3-hexene obtained in stage a) is dimerized in the presence of a dimerization catalyst to give a C₁₀₋₁₂-olefin mixture.

The resulting dimer olefin mixtures according to the invention preferably have an average degree of branching in the range from 1 to 2,5, particularly preferably 1 to 2.0, in particular 1 to 1.5 and specifically 1 to 1.2. The degree of branching of a pure olefin is defined here as the number of carbon atoms which are linked to three carbon atoms, plus two times the number of carbon atoms which are linked to 4 carbon atoms. The degree of branching of a pure olefin can be measured here readily following total hydrogenation to the alkane via ¹H NMR via the integration of the signals of the methyl groups relative to the methylene and methine protons.

For mixtures of olefins, the degrees of branching are weighted with the molar percentages, and thus an average degree of branching is calculated.

The molar fractions are determined here ideally by means of gas chromatography.

The type of branching in the olefin is preferably such that, following hydrogenation, less than 10%, preferably less than 5%, particularly preferably less than 1%, of alkanes are obtained which do not belong to the methyl-, dimethyl-, ethylmethyl- and diethylalkanes. This means that the branches are only methyl and ethyl branches.

According to a particularly preferred embodiment of the invention, the dimerization is carried out such that the catalysis produces directly the desired advantageous composition relative to the branching structures.

According to a further embodiment of the invention, the resulting C₁₀₋₁₂-olefins are removed and 5 to 30% by weight, preferably 5 to 20% by weight, in particular up to 10 to 20% by weight, based on the removed C₁₀₋₁₂-olefins, of low-boiling constituents of the C₁₀₋₁₂-olefins are removed. Low-boiling constituents is the term used for the fraction of the C₁₀₋₁₂-olefin mixture which, during distillation, passes over first or has the lowest boiling point. Said weight fraction thus corresponds to the fraction which, during distillation, passes over first and can thus be separated off. Removal can, however, also take place via any other suitable methods. In particular, fractional distillation is carried out. As a result of the separation carried out in accordance with the invention, the polybranched olefins are removed in part or preferably in their entirety from the C₁₀₋₁₂-olefin mixture. The removal can also be carried out such that at least 80%, preferably at least 90%, in particular at least 95% of the di- or polybranched olefins are separated off. In the C₁₀₋₁₂-olefin mixture at the end of stage b), the linear and singly branched olefins and possibly lower contents of polybranched olefins thus remain. Suitable separation methods and analytical methods for determining the content of polybranched olefins are known to the person skilled in the art.

Said embodiments can be combined with the addition of linear olefins in stage c), linear alkylbenzenes in stage d), linear alkylarylsulfonates in stage e) or combinations thereof. It is, however, also possible to dispense with an addition of such linear compounds.

If linear compounds are added in stages c), d) and/or e), then, according to one embodiment, it is possible to dispense with separating off low-boiling constituents in stage b).

In the dimerization mixture, <30, preferably <10% by weight of alkanes and <5% by weight of non-C₁₀₋₁₂-olefins may be present.

For the dimerization, preference is given to using the internal, linear pentenes and hexenes present in the metathesis product. Particular preference is given to the use of 3-hexene.

The dimerization can be carried out with homogeneous catalysis or heterogeneous catalysis. The homogeneously catalyzed dimerization can be varied within wide limits relative to the branching structures. As well as nickel systems, it is also possible to use, for example, Ti, Zr, Cr or Fe systems, which can be modified in a targeted manner via further cocatalysts and ligands.

The homogeneously catalyzed dimerization in the absence of transition metals is particularly preferably catalyzed with aluminum alkyls AlR₃. While these α-olefins react selectively to vinylidenes under very mild conditions, the corresponding reaction of internal olefins is also possible under more drastic conditions. Here too, dimers with a high vinylidene content are formed. The proportion of di- and triple-branched isomers is extremely low.

The AlR₃-catalyzed dimerization is preferably carried out at temperatures in the range from 150 to 300° C., particularly preferably 180 to 240° C., in particular 210 to 230° C., the catalyst is preferably separated off by distillation via the still and recycled to the catalysis. For the heterogeneous catalysis, use is expediently made of combinations of oxides of metals of subgroup VIII with aluminum oxide on support materials of silicon and titanium oxides, as are known, for example, from DE-A-43 39 713. The heterogeneous catalyst can be used in a fixed bed (then preferably in coarsely particulate form as 1 to 1.5 mm chips) or in suspended form (particle size 0.05 to 0.5 mm). The dimerization is carried out in the case of the heterogeneous procedure expediently at temperatures of from 80 to 200° C., preferably from 100 to 180° C., under the pressure prevailing at the reaction temperature, optionally also under a protective gas at a pressure above atmospheric pressure, in a closed system. To achieve optimum conversions, the reaction mixture is repeatedly cycled, a certain fraction of the circulating product being discharged and replaced by starting material continuously.

In the dimerization according to the invention, mixtures of monounsaturated hydrocarbons are obtained whose components predominantly have a chain length which is twice that of the starting olefins.

In C₁₂-olefin mixtures prepared according to the invention, the main chain preferably carries methyl or ethyl groups on the branching points.

The olefin mixtures obtainable by the above process (cf. WO 00/39058) represent valuable intermediates, in particular for the preparation, described below, of branched alkyl aromatics for the preparation of surfactants.

Stage c)

In stage c) the C₁₀₋₁₂-olefin mixture obtained in stage b) is reacted with an aromatic hydrocarbon in the presence of an alkylating catalyst to form alkyl aromatic compounds.

The C₁₀₋₁₂-olefin mixture used in stage c) has an optimum structure/linearity. This means that the degree of branching and the type of branching are optimally chosen in order to obtain advantageous alkyl aromatic compounds in stage c). The adjustment of the C₁₀₋₁₂-olefin mixture to be used optimally in stage c) can take place by admixing linear olefins. Preferably, however, more highly branched olefins are separated off instead of an admixing of linear olefins. Particularly preferably, in the dimerization, a suitable catalyst is combined with a suitable processing method in order to obtain the optimum C₁₀₋₁₂-olefin mixture. In this processing method, the desired structures are obtained directly in the alkylation. In this case, it is possible to dispense with the admixing of linear olefins and the removal of more highly branched olefins. Combinations of the processing methods described are also possible.

If in stage b) a removal of low-boiling components is carried out, in stage c) 0 to 60% by weight, preferably 0 to 50% by weight, in particular 0 to 30% by weight, based on the C₁₀₋₁₂-olefin mixtures obtained in stage b), of linear olefins can be added if desired. If linear olefins are added, their amount is at least 1% by weight, preferably at least 5% by weight, in particular at least 10% by weight.

If, according to the second embodiment of the invention, no removal of low-boiling components is carried out in stage b), in at least one of stages c), d) and e) 5 to 60% by weight, in each case based on the mixtures obtained in the previous stage, of the linear compounds are added. This means that in stage c) additionally linear olefins are added and/or in stage d) additionally linear alkylbenzenes are added and/or in stage e) additionally linear alkylarylsulfonates are added. Thus, linear compounds can be added in each of the stages c), d) and e), and also in individual stages or two of these stages. In stage c) 5 to 60% by weight, preferably 10 to 50% by weight, in particular 10 to 30% by weight, based on the C₁₀₋₁₂-olefin mixtures obtained in stage b), of linear olefins can thus be added.

Based on stages c), d) and e) overall, preferably at most 60% by weight, particularly preferably at most 40% by weight, in particular at most 30% by, weight, of the linear compounds are added. If this maximum amount is already achieved by the addition in one of these stages, in the other stages an addition of linear compounds is dispensed with.

As a result of the addition of the linear compounds, the profile of properties of the alkylarylsulfonates can be adapted over and above the advantageous synthesis sequence to the respective desired field of application and the profile of requirements.

The lower limits mentioned in each case can be combined with the upper limits mentioned in each case to give ranges which are possible according to the invention.

Thus, preference is given to using an alkylation catalyst which leads to alkyl aromatic compounds which have one to three carbon atoms with a H/C index of 1 in the alkyl radical.

The alkylation can in principle be carried out in the presence of any alkylation catalysts.

Although AlCl₃ and HF can be used in principle, heterogeneous or shape-selective catalysts offer advantages. For reasons of plant safety and environmental protection, preference is nowadays given to solid catalysts, which include, for example, the fluorinated Si/Al catalyst used in the DETAL process, a number of shape-selective catalysts and supported metal oxide catalysts, and also phyllosilicates and clays.

In the choice of catalyst, despite the large influence of the feedstock used, an important aspect is to minimize compounds formed by the catalyst which are notable for the fact that they include C atoms with a H/C index of 0 in the alkyl radical. Furthermore, compounds should be formed which on average have 1 to 3 C atoms with a H/C index of 1 in the alkyl radical. This can be achieved, in particular, through the choice of suitable catalysts which, on the one hand, suppress the formation of the undesired products as a result of their geometry, but on the other hand permit an adequate reaction rate.

The alkyl aromatic compounds according to the invention have a characteristic content of primary, secondary, tertiary and quaternary carbon atoms in the alkyl radical (side chain). This is reflected in the number of carbon atoms in the alkyl radical with a H/C index of from 0 to 3. The H/C index defines here the number of protons per carbon atom in the alkyl radical. Preferably, the mixtures of alkyl aromatic compounds according to the invention have only a small fraction of carbon atoms in the alkyl radical with a H/C index of 0. Preferably, the fraction of carbon atoms in the alkyl radical with a H/C index of 0 is, from an average of all compounds, <15%, particularly preferably <10%. The fraction of carbon atoms in the alkyl radical with a H/C index of 0 which are simultaneously bonded to the aromatics is ≧80%, preferably ≧90%, particularly preferably ≧95% of all carbon atoms in the alkyl radical with an H/C index of 0.

Preferably, the mixtures of alkyl aromatic compounds according to the invention have on average 1 to 3, preferably 1 to 2.5, particularly preferably 1 to 2, carbon atoms in the side chain (i.e. without counting the aromatic carbon atoms) with a H/C index of 1. The proportion of compounds with three carbon atoms of this type is preferably <30%, particularly preferably <20%, in particular <10%.

The fraction of carbon atoms which have a certain H/C index can be controlled through appropriate choice of the catalyst used. Preferredly used catalysts with which advantageous H/C distributions are achieved are mordenite, β-zeolite, L-zeolite, MCM-58, MCM-68 and faujasite. Particular preference is given to mordenite and faujasite.

In choosing the catalysts, their tendency with regard to deactivation must moreover be taken into consideration. One-dimensional pore systems in most cases have the disadvantage of rapid blockage of the pores by degradation products or synthesis products from the process. Catalysts with polydimensional pore systems are therefore preferred.

The catalysts used can be of natural or synthetic origin, whose properties can be adjusted by methods known from the literature (e.g. ion exchange, steaming, blocking of acid centers, washing out of extralattice species, etc.) to a certain extent. It is important for the present invention that the catalysts at least partially have acidic character.

Depending on the type of application, the catalysts are either in the form of powders or moldings. The linkages of the matrices of the moldings ensured adequate mechanical stability, although free access of the molecules to the active constituents of the catalysts is to be ensured through adequate porosity of the matrices. The preparation of such moldings is known in the literature and is carried out in accordance with the prior art.

Preferred Reaction Procedure

The alkylation is carried out by reacting the aromatic (the aromatic mixture) and the olefin (mixture) in a suitable reaction zone by bringing them into contact with the catalyst, working up the reaction mixture after the reaction and thus obtaining the products of value.

Suitable reaction zones are, for example, tubular reactors or stirred-tank reactors. If the catalyst is in solid form, then it can be used either as a slurry, as a fixed bed or as a fluidized bed. Execution as a catalytic distillation is also possible.

The reactants are either in the liquid and/or in the gaseous state.

The reaction temperature is chosen such that on the one hand as complete as possible a conversion of the olefin takes place and on the other hand the fewest possible by-products are formed. The choice of temperature control also depends decisively on the catalyst chosen. Reaction temperatures between 50° C. and 500° C. (preferably 80 to 350° C., particularly preferably 80-250° C.) can be used. The pressure of the reaction is governed by the procedure chosen (reactor type) and is between 0.1 and 100 bar, the weight hourly space velocity (WHSV) is chosen between 0.1 and 100. The procedure is generally carried out under intrinsic pressure (the vapor pressure of the system) or above.

The reactants can optionally be diluted with inert substances. Inert substances are preferably paraffins.

The molar ratio of aromatic:olefin is usually adjusted between 1:1 and 100:1 (preferably 2:1-20:1).

Aromatic Feed Substances

Possible substances are all aromatic hydrocarbons of the formula Ar—R, where Ar is a monocyclic or bicyclic aromatic hydrocarbon radical, and R is chosen from H, C₁₋₅ preferably C₁₋₃-alkyl, OH, OR etc., preferably H or C₁₋₃-alkyl. Preference is given to benzene and toluene.

Stage d)

In stage d) the alkyl aromatic compounds obtained in stage c) are sulfonated and neutralized to give alkylarylsulfonates.

The alkylaryls are converted to alkylarylsulfonates by
1) sulfonation (e.g. with SO₃, oleum, chlorosulfonic acid, etc., preferably with SO₃) and
2) neutralization (e.g. with Na, K, NH4, Mg compounds, preferably with Na compounds).

Sulfonation and neutralization are described adequately in the literature and are carried out in accordance with the prior art. The sulfonation is preferably carried out in a falling-film reactor, but can also take place in a stirred-tank reactor. The sulfonation with SO₃ is preferred over the sulfonation with oleum.

Mixtures

The compounds prepared by processes described above are either further processed as they are, or mixed beforehand with linear alkylaryls and then passed to further processing. In order to simplify this process, it may also be advisable to mix the raw materials which are used for the preparation of the abovementioned other alkylaryls directly with the raw materials of the present process and then to carry out the process according to the invention. Thus, for example, as described, the mixing of slightly branched olefin streams from the process according to the invention with linear olefins is advisable. Mixtures of the alkylarylsulfonic acids or of the alkylarylsulfonates can also be used. The mixings are always carried out with regard to the optimization of the product quality of the surfactants prepared from the alkylaryl.

In stage d) linear alkylbenzenes can additionally be added prior to the sulfonation. Their amount is 0 to 60% by weight, preferably 0 to 50% by weight, in particular 0 to 30% by weight. If no removal of low-boiling components is carried out in stage b), and no addition of linear compounds takes place in stages c) and e), the minimum amount is 5% by weight, preferably 10% by weight. Reference is made to the above statements regarding the total amount of the linear compounds added. In the linear alkylbenzenes, the chain length of the alkyl radicals preferably corresponds to the chain length of the alkyl radicals as is obtained from stage c) in the alkyl aromatic compounds. Preferably linear (C₁₀-alkyl)benzenes are added to (C₁₀-alkyl)benzenes and correspondingly linear (C₁₋₂-alkyl)benzenes are added to (C₁₋₂-alkyl)benzenes.

An exemplary overview of alkylation, sulfonation, neutralization is given, for example, in “Alkylarylsulfonates: History, Manufacture, Analysis and Environmental Properties” in Surf. Sci. Ser. 56 (1996) Chapter 2, Marcel Dekker, New York and references contained therein.

Stage e)

In stage e) the alkylarylsulfonates present in stage d) can additionally be mixed with linear alkylarylsulfonates.

In stage e) preferably 0 to 60% by weight, particularly preferably 0 to 50% by weight, in particular 0 to 30% by weight, of linear alkylarylsulfonates are added. If no removal of low-boiling constituents takes place in stage b), and no addition of linear compounds takes place in stages c) and d), the minimum amount is preferably 5% by weight, preferably at least 10% by weight. Reference is made to the abovementioned preferred total amounts for the addition of linear compounds.

All of the weight data refer in each case to the mixtures obtained in the preceding stage.

The invention also provides alkylarylsulfonates obtainable by a process as described above.

The alkylarylsulfonates according to the invention are preferably used as surfactants, in particular in detergents and cleaners. The invention also provides a detergent or cleaner comprising, as well as customary ingredients, alkylarylsulfonates as described above.

Nonexclusive examples of customary ingredients of the detergents and cleaners according to the invention are listed, for example, in WO 02/44114 and WO 02/14266.

The invention is illustrated in more detail by reference to the examples below.

### EXAMPLE 1

A butadiene-free C₄ fraction with a total butene content of 84.2% by weight and a molar ratio of 1-butene to 2-butenes of 1:1.06 is passed continuously, at 40° C. and 10 bar, over a tubular reactor equipped with Re₂O₇/Al₂O₃ heterogeneous catalyst. The space velocity for the catalyst in the example is 4500 kg/m² h. The reaction discharge is separated by distillation and comprises the following components (data in percent by mass):

Ethene 1.15%; propene 18.9%, butanes 15.8%, 2-butenes 19.7%, 1-butene 13.3%, i-butene 1.0%, 2-pentene 19.4%, methylbutenes 0.45%, 3-hexene 10.3%.

2-Pentene and 3-hexene are obtained from the product by distillation in purities >99% by weight.

### EXAMPLE 2

Continuous Dimerization of 3-Hexene in a Fixed Bed Process

| | |
|---|---|
| Catalyst: | 50% NiO, 34% SiO₂, 13% TiO₂, 3% Al₂O₃ |
| | (as in DE 43 39 713) used as 1-1.5 mm chips (100 ml), |
| | conditioned for 24 h at 160° C. in N₂ |
| Reactor: | isothermal, 16 mm Ø reactor |
| WHSV: | 0.25 kg/l · h |
| Pressure: | 20 to 25 bar |
| Temperature: | 100 to 160° C. |

| Temperature | | 100 | 120 | 140 | 160 | 160 | | |
|---|---|---|---|---|---|---|---|---|
| (° C.) | | | | | | | | |
| Pressure | Feed- | 20 | 20 | 20 | 25 | 25 | Collected | C₁₂- |
| (bar) | stock | | | | | | — | Distillate |
| Operating hours | | 12 | 19 | 36 | 60 | 107 | Product | |
| Liquid produced | | 24 | 27 | 27 | 28 | 27 | | |
| (g/h) | | | | | | | | |
| Composition | | | | | | | | |
| (% by wt.) | | | | | | | | |
| C₆ | 99.9 | 68.5 | 52.7 | 43.6 | 57.0 | 73.2 | n.d. | 0.1 |
| C₇-C₁₁ | 0.1 | 0.2 | 0.2 | 0.3 | 0.2 | 0.2 | | — |
| C₁₂ | | 25.9 | 38.6 | 44.0 | 35.6 | 23.6 | | 99.9 |
| C₁₃+ | | 5.4 | 8.5 | 12.1 | 7.2 | 3.0 | | — |
| Conversion | | 31.4 | 47.2 | 56.4 | 42.9 | 26.7 | | |
| C₁₂-Selectivity | | 82.5 | 81.8 | 78.2 | 83.0 | 88.4 | | |
| (% by wt.) | | | | | | | | |
| S content in the | <1 | n.d. | n.d. | n.d. | n.d. | n.d. | n.d. | n.d. |
| liquid produced | | | | | | | | |
| (ppm) | | | | | | | | |

The collected product was distilled to a C₁₂ purity of 99.9% by weight.

### EXAMPLE 3

2-Pentene from the raffinate II metathesis was dimerized continuously over an Ni heterogeneous catalyst analogously to Example 2. Fractional distillation of the product resulted in a decene fraction with a purity of 99.5%.

### EXAMPLE 4

A mixture of 2-pentene and 3-hexene from the raffinate II metathesis was dimerized continuously analogously to Example 2 and Example 3. Fractional distillation of the product resulted in a decene/undecene/dodecene fraction with a purity of 99.5%.

### EXAMPLE 5

100 g of 3-hexene are reacted with 3 g of triethylaluminum. After 22 hours at a temperature of 220° C. the reaction is complete.

In the resulting product mixture, the molar ratio of dimer to trimer is 58. The proportion of 2-butyl-1-octene is 69%. The degree of branching is 1.03. The proportion of doubly and triply branched isomers is 2%.

### EXAMPLE 6

A tubular reactor located within a circulatory-air oven was charged with 32 g of catalyst chips (60% H-mordenite with SiO₂: Al₂O₃=24.5—shaped with 40% Pural® SB from Condea) of particle size 0.7-1 mm and activated for 6 hours at 500° C. The system was then cooled, flooded with a feed of benzene:dodecene from Example 5 (10:1 molar), reacted at a space velocity of 0.62 g/g_{cat}h, and a 10-fold higher circulatory stream was established. Finally, the reactor was heated to 180° C. (single liquid phase, 30 bar hydraulic pressure) and the content of starting materials and products in the exit stream was detected by means of GC with respect to time. The resulting C₁₋₈-alkylaryl mixture was purified by distillation and analyzed by means of coupled gas chromatography-mass spectrometry and ¹H/¹³C NMR.

1-7. (canceled)

8. A process for the preparation of alkylarylsulfonates by
a) reaction of a C₄-olefin mixture over a metathesis catalyst to prepare an olefin mixture comprising 2-pentene and/or 3-hexene, and optional removal of 2-pentene and/or 3-hexene,
b) dimerization of the 2-pentene and/or 3-hexene obtained in stage a) in the presence of a dimerization catalyst to give a mixture comprising C₁₀₋₁₂-olefins, removal of the C₁₀₋₁₂-olefins and removal of 5 to 30% by weight, based on the C₁₀₋₁₂-olefins removed, of low-boiling constituents of the C₁₀₋₁₂-olefins,
c) reaction of the C₁₀₋₁₂-olefin mixtures obtained in stage b) with an aromatic hydrocarbon in the presence of an alkylation catalyst to form alkyl aromatic compounds, where, prior to the reaction, 0 to 60% by weight, based on the C₁₀₋₁₂-olefin mixtures obtained in stage b), of linear olefins may additionally be added,
d) sulfonation of the alkyl aromatic compounds obtained in stage c) and neutralization to give alkylarylsulfonates, where, prior to the sulfonation, 0 to 60% by weight, based on the alkyl aromatic compounds obtained in stage c), of linear alkylbenzenes may additionally be added, if no admixing has taken place in stage c),
e) optional mixing of the alkylarylsulfonates obtained in stage d) with 0 to 60% by weight, based on the alkylarylsulfonates obtained in stage d), of linear alkylarylsulfonates, if no admixing has taken place in stages c) and d).

2. A process for the preparation of alkylarylsulfonates by
a) reaction of a C₄-olefin mixture over a metathesis catalyst to prepare an olefin mixture comprising 2-pentene and/or 3-hexene and optional removal of 2-pentene and/or 3-hexene,
b) dimerization of the 2-pentene and/or 3-hexene obtained in stage a) in the presence of a dimerization catalyst to give a mixture comprising C₁₀₋₁₂-olefins and optional removal of the C₁₀₋₁₂-olefins,
c) reaction of the C₁₀₋₁₂-olefin mixtures obtained in stage b) with an aromatic hydrocarbon in the presence of an alkylation catalyst to form alkyl aromatic compounds, where, prior to the reaction, additional linear olefins may be added,
d) sulfonation of the alkyl aromatic compounds obtained in stage c) and neutralization to give alkylarylsulfonates, where, prior to the sulfonation, linear alkylbenzenes may additionally be added,
e) optional mixing of the alkylarylsulfonates obtained in stage d) with linear alkylarylsulfonates,
where, in at least one of stages c), d) and e), 5 to 60% by weight, in each case based on the mixtures obtained in the previous stage, of the linear compounds are added and the sum of the additions is not more than 80% by weight.

10. The process as claimed in claim 8, wherein the metathesis catalyst in stage a) is chosen from compounds of a metal of group VIb, VIb or sub-group VIII of the Periodic Table of the Elements.

11. The process as claimed in claim 8, wherein, in stage b), a dimerization catalyst is used which comprises at least one element of sub-group VIII of the Periodic Table of the Elements.

12. The process as claimed in claim 8, wherein the dimer olefin mixtures obtained in stage b) have an average degree of branching in the range from 1 to 2.5.

13. The process as claimed in claim 8, wherein the dimer olefin mixtures obtained in stage b) have an average degree of branching in the range from 1 to 2.0.

14. The process as claimed in claim 8, wherein, in stage c), an alkylation catalyst is used which leads to alkyl aromatic compounds which have 1 to 3 carbon atoms with a H/C index of 1 in the alkyl radical.
